SEQUENCE LISTING (10 sequences)

SEQ ID NO: 1 - DNA, 4711 bp, Homo sapiens
  source 1..4711: mol_type = genomic DNA
agtgagctatggggacactactgcactgtagcctgggcaacagagcaagaccttgtctcaaaaatgtatatatattttgggctttttttcctaaaacgggaactacaacagcatatttgcgagctgatgagagtgacccagcagagagggaaatggatcagctctgttgaagatgcactggacaccagaacacgcccagcccctcaaccagtggccagagcagcacctggacgtctcctccaccaccccgtcgccggcccacaagttggagttgccccctgggggtcgccaacgctgccactacgcttgggcacacgacgacatctcagccctcactgcctccaacctcctaaagcgctatgcagagaagtactctggggtcttggattctccctacgagcgtccggccctgggcgggtacagcgacgcctccttcctcaacggcgccaaaggggatcccgagccctggccagggccggagccaccctaccccttggcctcactccacgaaggcctcccaggaaccaaatcgggcggtggcggcggttccggggccctggggggctccccagttttagccgggaacctccctgaacccctctacgccggcaatgcgtgcgggggcccatcggcggcgcccgagtacgcggccggctacggcggggggtacctggcgccgggttactgcgcgcagacgggcgccgcgctgcccccgccgcccccggccgcgctcctgcagcccccaccgcctccggggtacgggccctcagcgccgctgtacaactatcccgcagggggctacgcagcgcagcccggctatggcgcgctcccgccgcccccaggcccacccccggccccctacctgaccccgggcctgcccgcgcccacgcccctgcccgcgccggcaccgcccaccgcctatggcttccccacggccgcgccgggtgccgaatccgggctgtcgctgaagcgcaaggccgccgacgaggggcccgagggccgctaccgcaagtacgcgtacgagcccgccaaggcccccgtggctgacggagcctcctaccccgccgcggacaacggcgaatgtcggggcaacgggttccgggccaagccgccaggagccgcggaggaggcgtcgggcaagtacggtggcggcgtccccctcaaggtcctgggctcccccgtctacggcccgcaactggagccctttgaaaagttcccggagcgggccccggctcctcgtggggggttcgccgtgccgtcgggggagactcccaaaggcgtggaccctggggccctggagctggtgacgagcaagatggtggactgcgggcccccggtgcagtgggcggatgtggcgggccagggcgcgctcaaggcggcgctggaggaggagctggtgtggcccctgctcaggccgcccgcctacccgggcagcctgcgcccgccgcggaccgtcctgctctttgggccgcggggcgcgggcaaagcgctgctgggccgctgcctcgccacgcagctgggcgccacgctgttgcgcctgcgcggcgcgaccctggctgcgcccggcgccgccgagggcgcgcgcctcctccaggccgccttcgcggccgcgcgctgccgcccaccctccgtactcctcatcagcgagctagaggcgctgctccccgcccgggacgacggcgcggcggcagggggcgcgctgcaggtgccgctcctggcctgcctggacgggggctgcggcgcgggggctgacggcgtgctggttgtgggcaccacctcgcggcccgcggctctggacgaggcgacccgccggcgcttctctctccgcttctacgtggcgctgcccgacagcccggcccgcgggcagatcctgcagcgggcgctggcccagcagggctgcgcgctcagtgagcgggaactggcggcgctggtgcagggcacgcagggcttctctgggggcgagctggggcagctgtgccagcaggcggcggccggggcgggcctcccggggctgcagcgccccctctcctacaaggacctggaggcggcgctggccaaggtgggccctagggcctctgccaaggaactggactcgttcgtggagtgggacaaaatgtacggctccggacactgacggcgcgcgggggaggccgcgggagccgcagtccctccgtccccgccgcctccgcgtgggagggatgtcactgactaaacccggctggcaggggctggagtggtgaatgtgggatcggggacaggaggggtctgccggtggatattttttttttcgtgggaaggaaaatgcttctgccaggcagatgccatatgcgccgtgtactcaggtttttcctatttattgtggactggaagctcgccatctccgcccggcagaccgggcagatccggcatgggctggcacccggggccttaagaactcctgctctcttgccacaacgcttttgtctcctcgctatctgaatggcaccctccttctccctcactctctccatcccattctctgcattctcttggttttctctcccttttgctttgtcgctgacacccctgcccaccccatgctggccctgtttctctcctgcccctccctccccagctctccatccctcaccctctgtgcttctgtctccatccctggctctccagcgtccctggccttttggtccctgagctttaatgcctttccctgccttctgttcttatttggactgcagtggccctttgcaggagctctggaggcccaggggctgaggaggagggttacccctctacccatctgaaacctagggtctagggggatcaaggaaaaaaagtccccaaagaaggggaattttttgtttgtttttgaggggagatcccagaaatgtagcttgtttcatattttagtcttcttatttttgtaaaatgtgtagaatttgctgtttttctttttcttttgacaactcaggaagaaactgacctcagaaagaatgttagactttggctgctctcctgtgtgcccctcacacctgccccctcccccccactccatccaggggaccaaattctcccagacactcaaaaaatgagacttacggggaaggggagaggaagacccagaggcctcagtgaaaccccagctattcctggtcagaagcagaatgtattcctaagggcttcctccccagggccgaggcctaggcatgaatgtggggagtgggctgtggggtttgagagaagggaggccttattcctctcctgctgctccccaccccctgccccacccaacccctccgctgagtgttttctgtgaagggctatccagagttaggatgcccttgcccaattccttcctgagacccagaaggtagggtgggagggcccaaatgggaaggtgacctaagcagaaagtctccagaaaggtcatgtcccctggccctgccttggcagaggtccccagtgacttatgctaggaggattccatctgggtagacagtctggccacaaaatcagctactggacctcagccatctctgctggaggctctgaggaggagtgagcatccctcacttgtgggggctctgtgaggaaatgtgccttccccattcccccggagtcctaggtctggagctccagggctgggagagggtgagggagatgggcaggggtgttttctctgaccttgggggcttagtctcagtcctgcctgaactttccactaggcttggaacccttccaagaaccatatttctctccttcccaccaattttcccttgatgaggctttagcagtttgctcccaccacccccagcccatttcacaactctgatcttagtccaaagcaggggacacgcccccccaccaccactttttctctctcccatctcagcctcctgtgcagttccttgcctgcccgtgcatttcctagagtctactgcctcccccctggctgggagggtgtctgggggggatctttcaggggccctggcacccagggcctgtgctggcctaggagtgctgaccagaaggctgctctgttcccccccacccccgttgctttctggccccctctttggagccagccacccacagggctttggtgcctcagaagcagtgggctgccgggtcacagccgcaggctgcaaaagaccctcggagggagcatggagtgaggggttctctctcaggtgtgtatgtattggggggtgggggtgggtggagggtgtcagggaagttggggtgggatcccagccttcccttcaagaggcagggagctctgggaggtggagtccccaccgctttctctactaggctcctcctgttccccaggcttggggagctttgcacaaggagactgcccccagcctagtggcacctacctcatgggctctggggcaggtaggggaagggccagtccagctctggtaatgctggggggaggcataccaaagaatccaggggcagggagtggggagggtgacttccgagctggcctctccccttcctctacccagactggggctgggatcctctcctcccgctgtaaccatttctacctcattttgctgcgtgttgtacatggacgtatttatctcctgtctgacgatgctctgcagttgtggtctgtctacctcagaagagactgtattttaaaagaaagtattacacagtattaaagcgatgacatgtggtttgcaaaaaaaaaaaaaaaaaa

SEQ ID NO: 2 - protein, 653 aa, Homo sapiens
MHWTPEHAQPLNQWPEQHLDVSSTTPSPAHKLELPPGGRQRCHYAWAHDDISALTASNLLKRYAEKYSGVLDSPYERPALGGYSDASFLNGAKGDPEPWPGPEPPYPLASLHEGLPGTKSGGGGGSGALGGSPVLAGNLPEPLYAGNACGGPSAAPEYAAGYGGGYLAPGYCAQTGAALPPPPPAALLQPPPPPGYGPSAPLYNYPAGGYAAQPGYGALPPPPGPPPAPYLTPGLPAPTPLPAPAPPTAYGFPTAAPGAESGLSLKRKAADEGPEGRYRKYAYEPAKAPVADGASYPAADNGECRGNGFRAKPPGAAEEASGKYGGGVPLKVLGSPVYGPQLEPFEKFPERAPAPRGGFAVPSGETPKGVDPGALELVTSKMVDCGPPVQWADVAGQGALKAALEEELVWPLLRPPAYPGSLRPPRTVLLFGPRGAGKALLGRCLATQLGATLLRLRGATLAAPGAAEGARLLQAAFAAARCRPPSVLLISELEALLPARDDGAAAGGALQVPLLACLDGGCGAGADGVLVVGTTSRPAALDEATRRRFSLRFYVALPDSPARGQILQRALAQQGCALSERELAALVQGTQGFSGGELGQLCQQAAAGAGLPGLQRPLSYKDLEAALAKVGPRASAKELDSFVEWDKMYGSGH

SEQ ID NO: 3 - RNA, 21 nt, Homo sapiens
  source 1..21: mol_type = other RNA
ttacacagtattaaagcgatt

SEQ ID NO: 4 - RNA, 21 nt, Homo sapiens
  source 1..21: mol_type = other RNA
tcgctttaatactgtgtaatt

SEQ ID NO: 5 - RNA, 21 nt, Homo sapiens
  source 1..21: mol_type = other RNA
catctgaaacctagggtcttt

SEQ ID NO: 6 - RNA, 21 nt, Homo sapiens
  source 1..21: mol_type = other RNA
agaccctaggtttcagatgtt

SEQ ID NO: 7 - RNA, 21 nt, Homo sapiens
  source 1..21: mol_type = other RNA
gtgacttatgctaggaggatt

SEQ ID NO: 8 - RNA, 21 nt, Homo sapiens
  source 1..21: mol_type = other RNA
tcctcctagcataagtcactt

SEQ ID NO: 9 - RNA, 21 nt, Homo sapiens
  source 1..21: mol_type = other RNA
ggtcagaagcagaatgtattt

SEQ ID NO: 10 - RNA, 21 nt, Homo sapiens
  source 1..21: mol_type = other RNA
atacattctgcttctgacctt

(19) United States
(12) Patent Application Publication
(10) Pub. No.: US 2023/0263820 A1
(43) Pub. Date: Aug. 24, 2023

(54) METHOD FOR ENHANCING RECOVERY OF COSMETIC LASER-TREATED SKIN

(71) Applicant: MICROCURES, INC., Santa Cruz, CA (US)

(72) Inventor: Brian O'Rourke, Boston, MA (US)

(73) Assignee: MICROCURES, INC., Santa Cruz, CA (US)

(21) Appl. No.: 17/963,962

(22) Filed: Oct. 11, 2022

Related U.S. Application Data
(63) Continuation of application No. 16/758,323, filed on Apr. 22, 2020, now abandoned.
(60) Provisional application No. 62/575,600, filed on Oct. 23, 2017.

(51) Int. Cl. A61K 31/7105; A61P 17/02; A61B 18/20; A61K 8/60; A61K 9/00; A61K 47/32; A61K 47/36; A61N 5/06; A61Q 19/08; C12N 15/113

(52) U.S. Cl. CPC A61K 31/7105; A61P 17/02; A61B 18/203; A61K 8/606; A61K 9/0014; A61K 9/0021; A61K 47/32; A61K 47/36; A61N 5/0616; A61Q 19/08; C12N 15/1137; A61B 2018/00458

(57) ABSTRACT

Methods of enhancing skin health recovery from a skin procedure comprising laser application to the skin using a siRNA or shRNA directed against a human Fidgetin like-2 nucleic acid.

### CROSS-REFERENCE TO RELATED APPLICATIONS

This application is a continuation of U.S. application Ser. No: 16/758,323, filed Apr. 22, 2020, which is a U.S. national stage application under 35 U.S.C. § 371 of International Appl. No: PCT/US2018/056007, filed Oct. 16, 2018, which claims priority to and the benefit of U.S. Provisional Appl. No: 62/575,600, filed Oct. 23, 2017, each of which is hereby incorporated by reference in its entirety.

### SEQUENCE LISTING INCORPORATION

The “.xml” Sequence Listing filed with this application by EFS and which is entitled P-86858-US1-SQL-15MAY23.xml.xml, is 14.7 kilobytes in size and which was created on May 11, 2023, is hereby incorporated by reference.

### BACKGROUND OF THE INVENTION

The disclosures of all publications, patents, patent application publications and books referred to in this application are hereby incorporated by reference in their entirety into the subject application to more fully describe the art to which the subject invention pertains.

Cosmetic procedures for the skin, such as lasabrasion, often involve an recovery period subsequent to treatment where the skin can be hypersensitive to light and/or touch, as well as redness and demarcation issues, and is uncomfortable for the subject. A method to reduce the recovery time and/or enhance the treatment effects is desirable.

The present invention addresses this need and identifies a novel target in promoting wound healing and provides therapies and assays based thereon.

### SUMMARY OF THE INVENTION

A method is provided of enhancing skin health recovery from a skin procedure comprising laser application to the skin, the method comprising directly administering to the skin that has undergone the procedure an amount of a siRNA or shRNA directed against a DNA or RNA encoding a human Fidgetin like-2 effective to enhance skin health recovery from a skin procedure comprising laser application to the skin.

Also provided is a method for increasing the rate of recovery of skin from a skin procedure comprising laser application to the skin, the method comprising directly administering to the skin that has undergone the procedure an amount of a siRNA or shRNA directed against a DNA or RNA encoding a human Fidgetin like-2 effective to increase the rate of recovery of skin recovering from a skin procedure comprising laser application to the skin.

Also provided is a method of promoting skin rejuvenation in skin subsequent to a skin procedure comprising laser application to the skin, the method comprising directly administering to the skin that has undergone the procedure an amount of a siRNA or shRNA directed against a DNA or RNA encoding a human Fidgetin like-2 effective to promote skin rejuvenation in skin subsequent to a skin procedure comprising laser application to the skin.

Also provided is a method comprising: treating a portion of a subject's skin by applying laser energy to the skin for cosmetic purposes; and administering, or directing the subject to administer, to the skin that has undergone the procedure an amount of a siRNA or shRNA directed against a DNA or RNA encoding a human Fidgetin like-2 effective to increase the rate of recovery of skin recovering from the treatment comprising applying laser energy to the skin.

Also provided is a method of reducing the visible appearance of a wrinkle in human skin comprising administering to the wrinkle an amount of a siRNA or shRNA directed against a DNA or RNA encoding a human Fidgetin like-2 effective to reduce the visible appearance of a wrinkle in human skin.

Also provided is a composition comprising (i) an amount of siRNA or shRNA is directed against an DNA encoding the human Fidgetin-like 2 effective to increase the rate of recovery of skin from a skin procedure comprising laser application to the skin contained (ii) in a microneedle array.

### BRIEF DESCRIPTION OF THE DRAWINGS

FIG. 1: FL2 siRNA improves wound healing in a mouse skin abrasion model. Age matched female BALB/c mice were shaved on their dorsal surface then treated with Nair to remove hair. Skin abrasions were made within a 1 cm by 1 cm region. After wounding the epidermal surface, mice were treated one time with either control nanoparticles containing scrambled siRNA or nanoparticles containing FL2 siRNA. After 5 days, the mice were sacrificed and their skin excised and sectioned for comparative H&E staining While controls showed significant wounding within the abrasion area, FL2 siRNA treated mice showed improved restoration of epidermal structure.

### DETAILED DESCRIPTION OF THE INVENTION

Herein is provided a method of enhancing skin health recovery from a skin procedure comprising laser application to the skin, the method comprising directly administering to the skin that has undergone the procedure an amount of a siRNA or shRNA directed against a DNA or RNA encoding a human Fidgetin like-2 effective to enhance skin health recovery from a skin procedure comprising laser application to the skin. Also provided is a method for increasing the rate of recovery of skin from a skin procedure comprising laser application to the skin, the method comprising directly administering to the skin that has undergone the procedure an amount of a siRNA or shRNA directed against a DNA or RNA encoding a human Fidgetin like-2 effective to increase the rate of recovery of skin recovering from a skin procedure comprising laser application to the skin.

Also provided is a method of promoting skin rejuvenation in skin subsequent to a skin procedure comprising laser application to the skin, the method comprising directly administering to the skin that has undergone the procedure an amount of a siRNA or shRNA directed against a DNA or RNA encoding a human Fidgetin like-2 effective to promote skin rejuvenation in skin subsequent to a skin procedure comprising laser application to the skin.

In an embodiment of the methods, the method promotes skin rejuvenation by increasing collagen I density in the skin.

In an embodiment of the methods, the method enhances skin health recovery by increasing collagen I density in the skin.

In an embodiment of the methods, the method promotes skin rejuvenation by increasing collagen I organization or improved linear orientation of the collagen fibers parallel to a dermoepidermal junction of the skin.

In an embodiment of the methods, the method enhances skin health recovery by increasing collagen I organization in the skin.

In an embodiment of the methods, the increased rate of recovery is a reduction in the extent of inflammation and/or an increased rate of inflammation reduction.

In an embodiment of the methods, the procedure is a cosmetic procedure. In an embodiment of the methods, the procedure is laser skin resurfacing. In an embodiment of the methods, the procedure is lasabrasion.

In an embodiment of the methods, the procedure is a medical procedure.

In an embodiment of the methods, the laser of the laser application is a non-ablative laser. In an embodiment of the methods, the laser of the laser application is an ablative laser.

In an embodiment of the methods, the Fidgetin like-2 comprises the amino acid set forth in SEQ ID NO:2

In an embodiment of the methods, the siRNA is administered. In an embodiment of the methods, the shRNA is administered. In an embodiment of the methods, the siRNA directed against a DNA or RNA encoding human Fidgetin-like 2 has at least one 2′ sugar modification.

In an embodiment of the methods, the shRNA directed against a DNA or RNA encoding human Fidgetin-like 2 has at least one 2′ sugar modification.

In an embodiment of the methods, the siRNA or shRNA is directed against an mRNA encoding the human Fidgetin-like 2.

In an embodiment of the methods, the siRNA or shRNA is directed against an DNA encoding the human Fidgetin-like 2.

In an embodiment of the methods, the siRNA comprises a sequence set forth in SEQ ID NOS:3, 4, 5, 6, 7, 8, 9, or 10.

Also provided is a method comprising:

treating a portion of a subject's skin by applying laser energy to the skin for cosmetic purposes; and
administering, or directing the subject to administer, to the skin that has undergone the procedure an amount of a siRNA or shRNA directed against a DNA or RNA encoding a human Fidgetin like-2 effective to increase the rate of recovery of skin recovering from the treatment comprising applying laser energy to the skin.

In an embodiment of the methods, the cosmetic purpose is to reduce the appearance of wrinkles, non-responsive skin after a facelift, aged or sun-damaged skin, skin liver spots, birthmark, wart, enlarged oil glands, port wine stains, hemangiomas, telangiectasias, or to change the appearance of skin complexion. In an embodiment of the methods, the birthmark is a linear epidermal nevus.

In an embodiment of the methods, the laser is a CO₂ laser.

In an embodiment of the methods, the laser is an erbium laser.

In an embodiment of the methods, the laser is a 595-nm PDL laser, 1,320-nm Nd:YAG laser, 1,064-nm Nd:YAG laser with long-pulse or Q-switched.

Also provided is a method of reducing the visible appearance of a wrinkle in human skin comprising administering to the wrinkle an amount of a siRNA or shRNA directed against a DNA or RNA encoding a human Fidgetin like-2 effective to reduce the visible appearance of a wrinkle in human skin.

In an embodiment, the Fidgetin like-2 comprises the amino acid set forth in SEQ ID NO:2. In an embodiment, the siRNA is administered. In an embodiment, the shRNA is administered. In an embodiment, the siRNA directed against a DNA or RNA encoding human Fidgetin-like 2 has at least one 2′ sugar modification. In an embodiment, the shRNA directed against a DNA or RNA encoding human Fidgetin-like 2 has at least one 2′ sugar modification. In an embodiment, the siRNA or shRNA is directed against an mRNA encoding the human Fidgetin-like 2. In an embodiment, the siRNA or shRNA is directed against an DNA encoding the human Fidgetin-like In an embodiment, the siRNA comprises a sequence set forth in SEQ ID NOS:3, 4, 5, 6, 7, 8, 9, or 10.

In an embodiment, siRNA or shRNA administration is begun on the same day as the laser skin treatment. In an embodiment, siRNA or shRNA administration is then continued every other day until the skin is healed.

In an embodiment, siRNA or shRNA administration is effected by administering liposomes containing the siRNA or shRNA.

In an embodiment, the method is used to enhance skin recovery subsequent to a:

chemical peel (e.g. superficial, medium-depth, and deep peels);
visible light device application;
intense pulsed light (IPL) application;
ablative or nonablative laser photo—rejuvenation;
radiofrequency (RF) application;
injectable skin biostimulation and/or rejuvenation procedure.

In an embodiment, the method is used to prevent dynamic wrinkles. In an embodiment, the method is used to correct static, anatomical wrinkles. In an embodiment, improvement is seen in wrinkle depth as measured using skin profilometry. This involves taking a mold of the face before and after treatment and reading those molds with a three-dimensional camera. (See, for example, Patel et al., Dermatol. Surg. (2002) 28: 942-945, hereby incorporated by reference.)

In an embodiment, the method is used to enhance skin recovery subsequent to a restoration (redistribution) of fat and/or volume loss procedure. In an embodiment, the method is used to enhance skin recovery subsequent to a skin augmentation and/or contouring procedure.

In an embodiment, the method is used to enhance skin recovery subsequent to a treatment to increase skin elasticity, increase skin smoothness, reduce skin fine lines, reduce signs of skin aging, reduce uneven skin tone, reduce acne, reduce skin hyperpigmentation, reduce skin discoloration, reduce skin sun spots or age spots, or reduce skin discoloration.

In an embodiment, the inhibitor of Fidgetin-like 2 is administered topically to the skin. In an embodiment, the inhibitor of Fidgetin-like 2 is administered from a reservoir that elutes the inhibitor, for example an eluting skin patch. In an embodiment, the inhibitor of Fidgetin-like 2 is administered from microneedle patch, wherein the microneedles deliver the inhibitor of Fidgetin-like 2, such as the siRNA, into the skin when placed on the skin or adhered onto the skin.

In an embodiment, the inhibitor of Fidgetin-like 2 is an siRNA or shRNA. In an embodiment, the nucleic acid is directed against a DNA encoding Fidgetin-like 2 or against an mRNA encoding Fidgetin-like 2.

In an embodiment of the method, the inhibitor of Fidgetin-like 2 is encapsulated in a nanoparticle. In an embodiment the nanoparticle is a liposomal nanoparticle.

In an embodiment, the Fidgetin-like 2 is human Fidgetin-like 2.

In an embodiment, the Fidgetin-like 2 comprises consecutive amino acid residues having the sequence set forth in SEQ ID NO:2.

The dosage of the inhibitor administered in treatment will vary depending upon factors such as the pharmacodynamic characteristics of a specific inhibitor and its mode and route of administration; the age, sex, metabolic rate, absorptive efficiency, health and weight of the recipient; the nature and extent of the symptoms; the kind of concurrent treatment being administered; the frequency of treatment with the inhibitor and the desired therapeutic effect.

A dosage unit of the inhibitor may comprise a single compound, or a mixture of the compound with one or more anti-infection compound(s) and/or cosmetic compounds.

In an embodiment, the siRNA (small interfering RNA) as used in the methods or compositions described herein comprises a portion which is complementary to an mRNA sequence encoding a Fidgetin-like 2 protein. In an embodiment, the Fidgetin-like 2 protein is a human Fidgetin-like 2 protein. In an embodiment, the mRNA is encoded by the DNA sequence NCBI Reference Sequence: NM_001013690.4 (SEQ ID NO:1), and the siRNA is effective to inhibit expression of Fidgetin-like 2 protein. In an embodiment, the Fidgetin-like 2 protein comprises consecutive amino acid residues having the sequence set forth in SEQ ID NO:2.

In an embodiment, the siRNA comprises a double-stranded portion (duplex). In an embodiment, the siRNA is 19-25 nucleotides in length. In an embodiment, the siRNA is 20-25 nucleotides in length. In an embodiment the siRNA comprises a 19-21 core RNA duplex with a one or two nucleotide 3′ overhang on, independently, either one or both strands. In an embodiment the siRNA comprises a 19-25 RNA duplex with a one or two nucleotide 3′ overhang on, independently, either one or both strands. The siRNA can be 5′ phosphorylated, or not, and may be modified with any of the known modifications in the art to improve efficacy and/or resistance to nuclease degradation. In an embodiment the siRNA can be administered such that it is transfected into one or more cells. In an embodiment, the siRNA is 5′ phosphorylated. In an embodiment, the whole length of the non-overlapping portion of the siRNA is fully complementary to a portion of a mRNA encoding a Fidgetin-like 2 protein.

In an embodiment, the 5′ terminal residue of a strand of the siRNA is phosphorylated. In an embodiment the 5′ terminal residue of the antisense strand of the siRNA is phosphorylated. In one embodiment, a siRNA of the invention comprises a double-stranded RNA wherein one strand of the double-stranded RNA is 80, 85, 90, 95 or 100% complementary to a portion of an RNA transcript of a gene encoding Fidgetin-like 2 protein. In an embodiment, the RNA transcript of a gene encoding Fidgetin-like 2 protein is an mRNA. In an embodiment, the Fidgetin-like 2 protein is a human Fidgetin-like 2 protein. In an embodiment, a siRNA of the invention comprises a double-stranded RNA wherein one strand of the RNA comprises a portion having a sequence the same as a portion of 18-25 consecutive nucleotides of an RNA transcript of a gene encoding Fidgetin-like 2 protein . In an embodiment, the Fidgetin-like 2 protein is a human Fidgetin-like 2 protein. In yet another embodiment, a siRNA of the invention comprises a double-stranded RNA wherein both strands of RNA are connected by a non-nucleotide linker. Alternately, a siRNA of the invention can comprise a double-stranded RNA wherein both strands of RNA are connected by a nucleotide linker, such as a loop or stem loop structure. In an embodiment, both of the strands of RNA are not connected by a nucleotide linker, such as a loop or stem loop structure.

In one embodiment, a single strand component of a siRNA of the invention is from 14 to 50 nucleotides in length. In another embodiment, a single strand component of a siRNA of the invention is 14, 15, 16, 17, 18, 19, 20, 21, 22, 23, 24, 25, 26, 27, or 28 nucleotides in length. In yet another embodiment, a single strand component of a siRNA of the invention is 21 nucleotides in length. In yet another embodiment, a single strand component of a siRNA of the invention is 22 nucleotides in length. In yet another embodiment, a single strand component of a siRNA of the invention is 23 nucleotides in length. In one embodiment, a siRNA of the invention is from 28 to 56 nucleotides in length. In another embodiment, a siRNA of the invention is 40, 41, 42, 43, 44, 45, 46, 47, 48, 49, 50, 51, or 52 nucleotides in length.

In another embodiment, an siRNA of the invention comprises at least one 2′-sugar modification. In another embodiment, an siRNA of the invention comprises at least one nucleic acid base modification. In another embodiment, an siRNA of the invention comprises at least one phosphate backbone modification. In embodiments, an siRNA of the invention comprises at least one 2′-0-methyl modification. In embodiments, an siRNA of the invention comprises at least one phosphorodithioate (PS2).

As used herein, “at least one” means one or more.

In one embodiment, RNAi inhibition of Fidgetin-like 2 protein is effected by a short hairpin RNA (“shRNA”). The shRNA can be introduced into the appropriate cell by transduction with a vector. In an embodiment, the vector is a lentiviral vector. In an embodiment, the vector comprises a promoter. In an embodiment, the promoter is a U6 or H1 promoter. In an embodiment the shRNA encoded by the vector is a first nucleotide sequence ranging from 19-29 nucleotides complementary to the target gene/mRNA, in the present case the mRNA encodes Fidgetin-like 2 protein. In an embodiment the Fidgetin-like 2 protein is a human Fidgetin-like 2 protein. In an embodiment the shRNA encoded by the vector also comprises a short spacer of 4-15 nucleotides (a loop, which does not hybridize) and a 19-29 nucleotide sequence that is a reverse complement of the first nucleotide sequence. In an embodiment the siRNA resulting from intracellular processing of the shRNA has overhangs of 1 or 2 nucleotides. In an embodiment the siRNA resulting from intracellular processing of the shRNA overhangs has two 3′ overhangs. In an embodiment the overhangs are UU.

In an embodiment, the FL2 is encoded by NCBI Reference Sequence:

| NM_001013690.4 (nucleic acid encoding Human Fidgetin-like 2) | |
|---|---|
| (SEQ ID NO: 1) | |
| 1 | agtgagctat ggggacacta ctgcactgta gcctgggcaa cagagcaaga ccttgtctca |
| 61 | aaaatgtata tatattttgg gctttttttc ctaaaacggg aactacaaca gcatatttgc |
| 121 | gagctgatga gagtgaccca gcagagaggg aaatggatca gctctgttga agatgcactg |
| 181 | gacaccagaa cacgcccagc ccctcaacca gtggccagag cagcacctgg acgtctcctc |
| 241 | caccaccccg tcgccggccc acaagttgga gttgccccct gggggtcgcc aacgctgcca |
| 301 | ctacgcttgg gcacacgacg acatctcagc cctcactgcc tccaacctcc taaagcgcta |
| 361 | tgcagagaag tactctgggg tcttggattc tccctacgag cgtccggccc tgggcgggta |
| 421 | cagcgacgcc tccttcctca acggcgccaa aggggatccc gagccctggc cagggccgga |
| 481 | gccaccctac cccttggcct cactccacga aggcctccca ggaaccaaat cgggcggtgg |
| 541 | cggcggttcc ggggccctgg ggggctcccc agttttagcc gggaacctcc ctgaacccct |
| 601 | ctacgccggc aatgcgtgcg ggggcccatc ggcggcgccc gagtacgcgg ccggctacgg |
| 661 | cggggggtac ctggcgccgg gttactgcgc gcagacgggc gccgcgctgc ccccgccgcc |
| 721 | cccggccgcg ctcctgcagc ccccaccgcc tccggggtac gggccctcag cgccgctgta |
| 781 | caactatccc gcagggggct acgcagcgca gcccggctat ggcgcgctcc cgccgccccc |
| 841 | aggcccaccc ccggccccct acctgacccc gggcctgccc gcgcccacgc ccctgcccgc |
| 901 | gccggcaccg cccaccgcct atggcttccc cacggccgcg ccgggtgccg aatccgggct |
| 961 | gtcgctgaag cgcaaggccg ccgacgaggg gcccgagggc cgctaccgca agtacgcgta |
| 1021 | cgagcccgcc aaggcccccg tggctgacgg agcctcctac cccgccgcgg acaacggcga |
| 1081 | atgtcggggc aacgggttcc gggccaagcc gccaggagcc gcggaggagg cgtcgggcaa |
| 1141 | gtacggtggc ggcgtccccc tcaaggtcct gggctccccc gtctacggcc cgcaactgga |
| 1201 | gccctttgaa aagttcccgg agcgggcccc ggctcctcgt ggggggttcg ccgtgccgtc |
| 1261 | gggggagact cccaaaggcg tggaccctgg ggccctggag ctggtgacga gcaagatggt |
| 1321 | ggactgcggg cccccggtgc agtgggcgga tgtggcgggc cagggcgcgc tcaaggcggc |
| 1381 | gctggaggag gagctggtgt ggcccctgct caggccgccc gcctacccgg gcagcctgcg |
| 1441 | cccgccgcgg accgtcctgc tctttgggcc gcggggcgcg ggcaaagcgc tgctgggccg |
| 1501 | ctgcctcgcc acgcagctgg gcgccacgct gttgcgcctg cgcggcgcga ccctggctgc |
| 1561 | gcccggcgcc gccgagggcg cgcgcctcct ccaggccgcc ttcgcggccg cgcgctgccg |
| 1621 | cccaccctcc gtactcctca tcagcgagct agaggcgctg ctccccgccc gggacgacgg |
| 1681 | cgcggcggca gggggcgcgc tgcaggtgcc gctcctggcc tgcctggacg ggggctgcgg |
| 1741 | cgcgggggct gacggcgtgc tggttgtggg caccacctcg cggcccgcgg ctctggacga |
| 1801 | ggcgacccgc cggcgcttct ctctccgctt ctacgtggcg ctgcccgaca gcccggcccg |
| 1861 | cgggcagatc ctgcagcggg cgctggccca gcagggctgc gcgctcagtg agcgggaact |
| 1921 | ggcggcgctg gtgcagggca cgcagggctt ctctgggggc gagctggggc agctgtgcca |
| 1981 | gcaggcggcg gccggggcgg gcctcccggg gctgcagcgc cccctctcct acaaggacct |
| 2041 | ggaggcggcg ctggccaagg tgggccctag ggcctctgcc aaggaactgg actcgttcgt |
| 2101 | ggagtgggac aaaatgtacg gctccggaca ctgacggcgc gcgggggagg ccgcgggagc |
| 2161 | cgcagtccct ccgtccccgc cgcctccgcg tgggagggat gtcactgact aaacccggct |
| 2221 | ggcaggggct ggagtggtga atgtgggatc ggggacagga ggggtctgcc ggtggatatt |
| 2281 | ttttttttcg tgggaaggaa aatgcttctg ccaggcagat gccatatgcg ccgtgtactc |
| 2341 | aggtttttcc tatttattgt ggactggaag ctcgccatct ccgcccggca gaccgggcag |
| 2401 | atccggcatg ggctggcacc cggggcctta agaactcctg ctctcttgcc acaacgcttt |
| 2461 | tgtctcctcg ctatctgaat ggcaccctcc ttctccctca ctctctccat cccattctct |
| 2521 | gcattctctt ggttttctct cccttttgct ttgtcgctga cacccctgcc caccccatgc |
| 2581 | tggccctgtt tctctcctgc ccctccctcc ccagctctcc atccctcacc ctctgtgctt |
| 2641 | ctgtctccat ccctggctct ccagcgtccc tggccttttg gtccctgagc tttaatgcct |
| 2701 | ttccctgcct tctgttctta tttggactgc agtggccctt tgcaggagct ctggaggccc |
| 2761 | aggggctgag gaggagggtt acccctctac ccatctgaaa cctagggtct agggggatca |
| 2821 | aggaaaaaaa gtccccaaag aaggggaatt ttttgtttgt ttttgagggg agatcccaga |
| 2881 | aatgtagctt gtttcatatt ttagtcttct tatttttgta aaatgtgtag aatttgctgt |
| 2941 | ttttcttttt cttttgacaa ctcaggaaga aactgacctc agaaagaatg ttagactttg |
| 3001 | gctgctctcc tgtgtgcccc tcacacctgc cccctccccc ccactccatc caggggacca |
| 3061 | aattctccca gacactcaaa aaatgagact tacggggaag gggagaggaa gacccagagg |
| 3121 | cctcagtgaa accccagcta ttcctggtca gaagcagaat gtattcctaa gggcttcctc |
| 3181 | cccagggccg aggcctaggc atgaatgtgg ggagtgggct gtggggtttg agagaaggga |
| 3241 | ggccttattc ctctcctgct gctccccacc ccctgcccca cccaacccct ccgctgagtg |
| 3301 | ttttctgtga agggctatcc agagttagga tgcccttgcc caattccttc ctgagaccca |
| 3361 | gaaggtaggg tgggagggcc caaatgggaa ggtgacctaa gcagaaagtc tccagaaagg |
| 3421 | tcatgtcccc tggccctgcc ttggcagagg tccccagtga cttatgctag gaggattcca |
| 3481 | tctgggtaga cagtctggcc acaaaatcag ctactggacc tcagccatct ctgctggagg |
| 3541 | ctctgaggag gagtgagcat ccctcacttg tgggggctct gtgaggaaat gtgccttccc |
| 3601 | cattcccccg gagtcctagg tctggagctc cagggctggg agagggtgag ggagatgggc |
| 3661 | aggggtgttt tctctgacct tgggggctta gtctcagtcc tgcctgaact ttccactagg |
| 3721 | cttggaaccc ttccaagaac catatttctc tccttcccac caattttccc ttgatgaggc |
| 3781 | tttagcagtt tgctcccacc acccccagcc catttcacaa ctctgatctt agtccaaagc |
| 3841 | aggggacacg cccccccacc accacttttt ctctctccca tctcagcctc ctgtgcagtt |
| 3901 | ccttgcctgc ccgtgcattt cctagagtct actgcctccc ccctggctgg gagggtgtct |
| 3961 | gggggggatc tttcaggggc cctggcaccc agggcctgtg ctggcctagg agtgctgacc |
| 4021 | agaaggctgc tctgttcccc cccacccccg ttgctttctg gccccctctt tggagccagc |
| 4081 | cacccacagg gctttggtgc ctcagaagca gtgggctgcc gggtcacagc cgcaggctgc |
| 4141 | aaaagaccct cggagggagc atggagtgag gggttctctc tcaggtgtgt atgtattggg |
| 4201 | gggtgggggt gggtggaggg tgtcagggaa gttggggtgg gatcccagcc ttcccttcaa |
| 4261 | gaggcaggga gctctgggag gtggagtccc caccgctttc tctactaggc tcctcctgtt |
| 4321 | ccccaggctt ggggagcttt gcacaaggag actgccccca gcctagtggc acctacctca |
| 4381 | tgggctctgg ggcaggtagg ggaagggcca gtccagctct ggtaatgctg gggggaggca |
| 4441 | taccaaagaa tccaggggca gggagtgggg agggtgactt ccgagctggc ctctcccctt |
| 4501 | cctctaccca gactggggct gggatcctct cctcccgctg taaccatttc tacctcattt |
| 4561 | tgctgcgtgt tgtacatgga cgtatttatc tcctgtctga cgatgctctg cagttgtggt |
| 4621 | ctgtctacct cagaagagac tgtattttaa aagaaagtat tacacagtat taaagcgatg |
| 4681 | acatgtggtt tgcaaaaaaa aaaaaaaaaa a. |

In an embodiment, the FL2 protein sequence comprises:

(SEQ ID NO: 2)
MHWTPEHAQPLNQWPEQHLDVSSTTPSPAHKLELPPGGRQRCHYAWAHD
DISALTASNLLKRYAEKYSGVLDSPYERPALGGYSDASFLNGAKGDPEP
WPGPEPPYPLASLHEGLPGTKSGGGGGSGALGGSPVLAGNLPEPLYAGN
ACGGPSAAPEYAAGYGGGYLAPGYCAQTGAALPPPPPAALLQPPPPPGY
GPSAPLYNYPAGGYAAQPGYGALPPPPGPPPAPYLTPGLPAPTPLPAPA
PPTAYGFPTAAPGAESGLSLKRKAADEGPEGRYRKYAYEPAKAPVADGA
SYPAADNGECRGNGFRAKPPGAAEEASGKYGGGVPLKVLGSPVYGPQLE
PFEKFPERAPAPRGGFAVPSGETPKGVDPGALELVTSKMVDCGPPVQWA
DVAGQGALKAALEEELVWPLLRPPAYPGSLRPPRTVLLFGPRGAGKALL
GRCLATQLGATLLRLRGATLAAPGAAEGARLLQAAFAAARCRPPSVLLI
SELEALLPARDDGAAAGGALQVPLLACLDGGCGAGADGVLVVGTTSRPA
ALDEATRRRFSLRFYVALPDSPARGQILQRALAQQGCALSERELAALVQ
GTQGFSGGELGQLCQQAAAGAGLPGLQRPLSYKDLEAALAKVGPRASAK
ELDSFVEWDKMYGSGH.

In an embodiment, the FL2 is naturally occurring variant having 95% or greater identity with NCBI Reference Sequence: NM_001013690.4 (SEQ ID NO:1). In an embodiment, the FL2 is naturally occurring variant having 96% or greater identity with NCBI Reference Sequence: NM_001013690.4 (SEQ ID NO:1). In an embodiment, the FL2 is naturally occurring variant having 97% or greater identity with NCBI Reference Sequence: NM_001013690.4 (SEQ ID NO:1). In an embodiment, the FL2 is naturally occurring variant having 98% or greater identity with NCBI Reference Sequence: NM_001013690.4 (SEQ ID NO:1). In an embodiment, the FL2 is naturally occurring variant having 99% or greater identity with NCBI Reference Sequence: NM_001013690.4 (SEQ ID NO:1).

In embodiments, the siRNA comprise one of the following pairs of sense/antisense sequences:

Sense:
(SEQ ID NO: 3)
UUACACAGUAUUAAAGCGAUU
Antisense:
(SEQ ID NO: 4)
5′ UCGCUUUAAUACUGUGUAAUU;
or
Sense:
(SEQ ID NO: 5)
CAUCUGAAACCUAGGGUCUUU
Antisense:
(SEQ ID NO: 6)
5′ AGACCCUAGGUUUCAGAUGUU;
or
Sense:
(SEQ ID NO: 7)
GUGACUUAUGCUAGGAGGAUU
Antisense:
(SEQ ID NO: 8)
5′ UCCUCCUAGCAUAAGUCACUU;
or
Sense:
(SEQ ID NO: 9)
GGUCAGAAGCAGAAUGUAUUU
Antisense:
(SEQ ID NO: 10)
5′ AUACAUUCUGCUUCUGACCUU.

In an embodiment, the siRNA is double-stranded and comprises SEQ ID NO:3 and 4; SEQ ID NO:5 and 6; SEQ ID NO:7 and 8; or SEQ ID NO:9 and 10.

In an embodiment, the 5′ terminal residue of a strand of the siRNA is phosphorylated. In an embodiment the 5′ terminal residue of the antisense strand of the siRNA is phosphorylated. In an embodiment, the 5′ terminal residue of a strand of the siRNA is not phosphorylated. In an embodiment the 5′ terminal residue of the antisense strand of the siRNA is not phosphorylated.

In an embodiment the inhibitor of Fidgetin-like 2 is provided in a bulk-eroding system such as polylactic acid and glycolic acid (PLGA) copolymer based microspheres or microcapsules systems containing the inhibitor of Fidgetin-like 2. In an embodiment, blends of PLGA:ethylcellulose systems may be used as an appropriate carrier. A further medicament in accordance with this aspect of the invention may be formulated in a surface-eroding system wherein the inhibitor of Fidgetin-like 2 is embedded in an erodible matrix such as the poly(ortho) ester and polyanhydride matrices wherein the hydrolysis of the polymer is rapid. A medicament in accordance with this aspect of the invention may also be formulated by combining a pulsatile delivery system as described above and an immediate release system such as a lyophilized injectable composition described above.

In an embodiment, the inhibitor of FL2 is administered in a dissolving microneedle. In an embodiment, the dissolving microneedle comprises one or more of dextran, hyaluronic acid, and Polyvinylpyrrolidone/PVP.

In an embodiment, the inhibitor of FL2 is administered in a composition with polyethylenimine In a non-limiting example the polyethylenimine is 25 KDa PEI.

The inhibitor may be used in a composition with additives. Examples of suitable additives are sodium alginate, as a gelatinizing agent for preparing a suitable base, or cellulose derivatives, such as guar or xanthan gum, inorganic gelatinizing agents, such as aluminum hydroxide or bentonites (termed thixotropic gel-formers), polyacrylic acid derivatives, such as Carbopol®, polyvinylpyrrolidone, microcrystalline cellulose and carboxymethylcellulose. Amphiphilic low molecular weight and higher molecular weight compounds, and also phospholipids, are also suitable. The gels can be present either as water-based hydrogels or as hydrophobic organogels, for example based on mixtures of low and high molecular weight paraffin hydrocarbons and vaseline. The hydrophilic organogels can be prepared, for example, on the basis of high molecular weight polyethylene glycols. These gelatinous forms are washable. Hydrophobic organogels are also suitable. Hydrophobic additives, such as petroleum jelly, wax, oleyl alcohol, propylene glycol monostearate and/or propylene glycol monopalmitostearate, in particular isopropyl myristate can be included. In an embodiment the inhibitor is in a composition comprising one or more dyes, for example yellow and/or red iron oxide and/or titanium dioxide for the purpose of matching as regards color. Compositions may be in any suitable form including gels, lotions, balms, pastes, sprays, powders, bandages, wound dressing, emulsions, creams and ointments of the mixed-phase or amphiphilic emulsion systems (oil/water-water/oil mixed phase), liposomes and transfersomes or plasters/band aid-type coverings. Emulsifiers which can be employed in compositions comprising the inhibitor of Fidgetin-like 2 include anionic, cationic or neutral surfactants, for example alkali metal soaps, metal soaps, amine soaps, sulphurated and sulphonated compounds, invert soaps, higher fatty alcohols, partial fatty acid esters of sorbitan and polyoxyethylene sorbitan, e.g. lanette types, wool wax, lanolin or other synthetic products for preparing the oil/water and/or water/oil emulsions.

Compositions comprising the inhibitor of Fidgetin-like 2 can also comprise vaseline, natural or synthetic waxes, fatty acids, fatty alcohols, fatty acid esters, for example as monoglycerides, diglycerides or triglycerides, paraffin oil or vegetable oils, hydrogenated castor oil or coconut oil, hog fat, synthetic fats (for example based on caprylic acid, capric acid, lauric acid or stearic acid, such as Softisan®), or triglyceride mixtures, such as Miglyol®, can be used as lipids, in the form of fatty and/or oleaginous and/or waxy components for preparing the ointments, creams or emulsions of the compositions comprising the inhibitor of fidgetin-like 2 used in the methods described herein.

Osmotically active acids and alkaline solutions, for example hydrochloric acid, citric acid, sodium hydroxide solution, potassium hydroxide solution, sodium hydrogen carbonate, may also be ingredients of the compositions and, in addition, buffer systems, such as citrate, phosphate, tris buffer or triethanolamine, for adjusting the pH. It is possible to add preservatives as well, such as methyl benzoate or propyl benzoate (parabens) or sorbic acid, for increasing the stability.

Pastes, powders and solutions are additional forms of compositions comprising the inhibitor of Fidgetin-like 2 which can be applied topically. As consistency-imparting bases, the pastes frequently contain hydrophobic and hydrophilic auxiliary substances, preferably, however, hydrophobic auxiliary substances containing a very high proportion of solids. In order to increase dispersity, and also flowability and slipperiness, and also to prevent agglomerates, the powders or topically applicable powders can, for example, contain starch species, such as wheat or rice starch, flame-dispersed silicon dioxide or siliceous earth, which also serve as diluent.

In an embodiment, the compositions comprise further active ingredients suitable for accelerating recovery from a skin cosmetic procedure, for example one or more antibiotics, antiseptics, vitamins, anesthetics, antihistamines, anti-inflammatory agents, moisturizers, penetration-enhancing agents and/or anti-irritants. In an embodiment, the compositions do not comprise further active ingredients suitable for accelerating recovery from a skin cosmetic procedure, for example one or more antibiotics, antiseptics, vitamins, anesthetics, antihistamines, anti-inflammatory agents, moisturizers, penetration-enhancing agents and/or anti-irritants.

In an embodiment of the methods and compositions described herein the subject is a mammal In an embodiment the subject is human.

In one embodiment, excluded from the present invention is a method performed on skin which has a wound in the area of the skin being treated, i.e. a gross break or discontinuity in the structure of the skin tissue. Examples of wounds include ulcerations, bedsores, grazes, tears, cuts, and punctures.

Preferably the inhibitor is biomembrane-permeable or is conjugated or otherwise attached to a moiety which renders the inhibitor biomembrane-permeable.

A composition is provided comprising (i) an amount of siRNA or shRNA is directed against an DNA encoding the human Fidgetin-like 2 as described herein effective to increase the rate of recovery of skin from a skin procedure comprising laser application to the skin contained (ii) in a microneedle array.

In an embodiment, the microneedle array comprises a structure made of one or more of dextran, hyaluronic acid and PVP. In an embodiment, the composition comprises a polyethylenimine

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### Experimental Details

### Introduction

Cosmetic skin procedures are popular and widespread. However, patients can be self-conscious about, and/or in discomfort from, the subsequent recovery process. Methods to enhance the recovery process, and to reduce the visible aspects of the recovering skin, are desirable. Patients who want a quick recovery time can choose non-ablative or fractional resurfacing, although repeat treatments may be necessary to attain optimum results. The methods disclose herein reduce recovery times after treatment, which enables, for example, subjects to return to public appearance quicker or to choose more intense non-ablative (thereby reducing the overall number of treatments needed to meet a predetermined endpoint compared to those without applying the methods disclosed herein), or to chose an ablative treatment where previously only non-ablative was considered due to long recovery times needed for the former.

### EXAMPLE 1

Initially, inhibition of FL2 is performed in vivo in a Kunming type mouse model to determine its effect of collagen remodeling (e.g. see Liu et al., Lasers in Surgery and Medicine, 40: 13-19 (2006), incorporated by reference herein). Depilated skin is treated with laser, e.g. 595-nm PDL (pulsed dye laser) (10 ms), 1,320-nm Nd:YAG (neodymium-yttrium-aluminum garnet) laser (0.35 ms), 1,064-nm Nd:YAG lasers with long-pulsed (0.3ms), and Q-switched (5 ns). Laser-treated skin is subsequently treated at one location with siRNA or shRNA directed to FL2 and at a second location with control. The skin is then examined at the FL2 treatment site and control site at 1 hour, 1 day, 1 week, 3 weeks, 4 weeks, and 8 weeks after laser treatment. Skin treated with siRNA or shRNA directed to FL2 shows increased rate and extent of re-epithelialization of the skin compared to control. In addition, the topically applied siRNA or shRNA is effective to increase collagen density and organization in the skin compared to control.

### EXAMPLE 2

A portion of skin on a human, for example facial skin, that has been treated with a lasabrasion procedure is subsequently treated with a topically applied siRNA or shRNA which inhibits Fidgetin-like 2. The topically applied siRNA or shRNA is effective to increase the rate and extent of re-epithelialization of the skin compared to control. In addition, the topically applied siRNA or shRNA is effective to increase collagen density and organization in the skin compared to control. Moreover, the topically applied siRNA or shRNA is effective to accelerate the rate of visual healing of the skin relative to controls. Visual healing can be assessed as evaluation of tactile roughness, visual texture, wrinkles, blotchiness, skin tone evenness, radiance, and translucence, e.g. on a 5-point scale. Other suitable methods are set forth, for example, in Hillebrand et al. (British Journal of Dermatology (2010) 162: 647-654, hereby incorporated by reference, see: “improvement in the appearance of fine lines and wrinkles was measured by expert visual grading of high-resolution digital images using the rapid evaluation of anti-aging leads (REAL 3.0) system taken at baseline and at 8 and (in the cohort) 24 weeks”).

In addition, the topically applied siRNA or shRNA is effective to reduce inflammation of the skin relative to controls. This results in an improved quality of recovery for the subject since visible redness and inflammation is a primary cause of the effective recovery time for such cosmetic procedures. This treatment manifests itself in improved outcomes as measured by shorter healing times and/or reduced wrinkling, permitting subjects to return to work and public life more quickly than otherwise.

### EXAMPLE 3

A portion of skin on a human, which portion has one or more fine lines and/or wrinkles, is treated with a topically applied siRNA or shRNA which inhibits Fidgetin-like 2. The topically applied siRNA or shRNA is effective to improve the appearance of fine lines and/or wrinkles (see also, for example, methods used in Hillebrand et al., British Journal of Dermatology (2010) 162: 647-654, hereby incorporated by reference).

### EXAMPLE 4

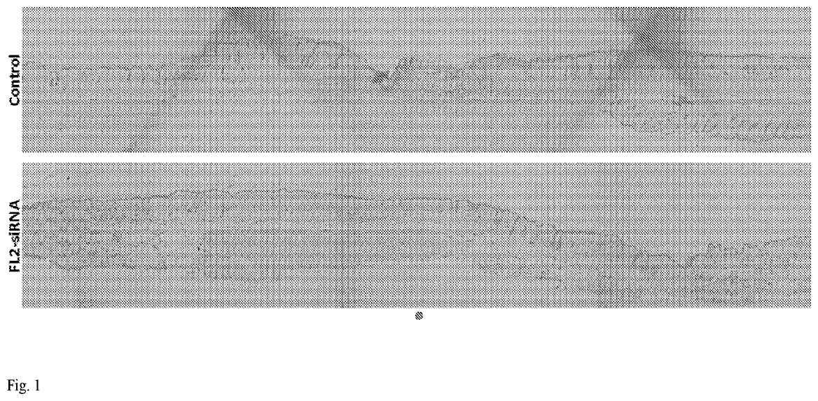

FL2 siRNA improves wound healing in a mouse skin abrasion model. Age matched female BALB/c mice were shaved on their dorsal surface then treated with Nair to remove hair. Skin abrasions were made within a 1 cm by 1 cm region. After wounding the epidermal surface, mice were treated one time with either control nanoparticles containing scrambled siRNA or nanoparticles containing FL2 siRNA. After 5 days, the mice were sacrificed and their skin excised and sectioned for comparative H&E staining. While controls showed significant wounding within the abrasion area, FL2 siRNA treated mouse showed improved restoration of epidermal structure as shown in FIG. 1.

1. A method of promoting skin rejuvenation, enhancing skin health recovery or increasing the rate of recovery of skin subsequent to a skin procedure comprising laser application to the skin, the method comprising directly administering to the skin that has undergone the procedure an amount of a siRNA or shRNA directed against a DNA or RNA encoding a human Fidgetin like-2 effective to promote skin rejuvenation, enhance skin health recovery or increase the rate of recovery of skin subsequent to a skin procedure comprising laser application to the skin.

2.-3. (canceled)

4. The method of claim 1, wherein the method promotes skin rejuvenation or skin health recovery by increasing collagen I density or organization in the skin.

5. (canceled)

6. The method of claim 1, wherein the method promotes skin rejuvenation by increasing collagen I organization, or improved linear orientation of the collagen fibers parallel to a dermoepidermal junction of the skin.

7. (canceled)

8. The method of claim 1, wherein the increased rate of recovery is a reduction in the extent of inflammation and/or an increased rate of inflammation reduction.

9. The method of claim 1, wherein the procedure is a cosmetic procedure.

10. The method of claim 9, wherein the procedure is laser skin resurfacing or lasabrasion.

11. (canceled)

12. The method of claim 1, wherein the procedure is a medical procedure.

13. The method of claim 1, wherein the laser of the laser application is a non-ablative laser or an ablative laser.

14. The method of claim 1, wherein the laser is a CO₂ laser, an erbium laser, a 595-nm PDL laser, a 1,320-nm Nd:YAG laser, or a 1,064-nm Nd:YAG laser with long-pulse or Q-switched.

15. The method of claim 1, wherein the Fidgetin like-2 comprises the amino acid sequence set forth in SEQ ID NO:2

16.-17.(canceled)

18. The method of claim 1, wherein the siRNA or shRNA directed against a DNA or RNA encoding human Fidgetin-like 2 has at least one 2′ sugar modification.

19.-21. (canceled)

22. The method of claim 1, wherein the siRNA comprises a sequence set forth in SEQ ID NOS:3, 4, 5, 6, 7, 8, 9, or 10.

23. A method comprising:
treating a portion of a subject's skin by applying laser energy to the skin for cosmetic purposes; and
administering, or directing the subject to administer, to the skin that has undergone the procedure an amount of a siRNA or shRNA directed against a DNA or RNA encoding a human Fidgetin like-2 effective to increase the rate of recovery of skin recovering from the treatment comprising applying laser energy to the skin.

24. The method of claim 23, wherein the cosmetic purpose is to reduce the appearance of wrinkles, non-responsive skin after a facelift, aged or sun-damaged skin, skin liver spots, birthmark, wart, enlarged oil glands, port wine stains, hemangiomas, telangiectasias, or to change the appearance of skin complexion.

25. The method of claim 24, wherein the birthmark is a linear epidermal nevus.

26. The method of claim 23, wherein the laser is a CO₂ laser, an erbium laser, a 595-nm PDL laser, a 1,320-nm Nd:YAG laser, or a 1,064-nm Nd:YAG laser with long-pulse or Q-switched.

27.-36. (canceled)

37. The composition of claim 38, wherein the siRNA comprises a sequence set forth in SEQ ID NOS:3, 4, 5, 6, 7, 8, 9, or 10.

38. A composition comprising (i) an amount of siRNA or shRNA is directed against an DNA encoding the human Fidgetin-like 2 effective to reduce the visible appearance of wrinkles or increase the rate of recovery of skin from a skin procedure comprising laser application to the skin, contained (ii) in a microneedle array.

39. The composition of claim 38, wherein the microneedle array comprises a structure made of one or more of dextran, hyaluronic acid and PVP.
